(19) United States
(12) Patent Application Publication
(10) Pub. No.: US 2020/0347437 A1
(43) Pub. Date: Nov. 5, 2020

(54) NUCLEIC ACID TESTS WITH TEMPORAL FLUORESCENT SIGNALS

(71) Applicant: HEWLETT-PACKARD DEVELOPMENT COMPANY, L.P., Spring, TX (US)

(72) Inventor: Rachel M. WHITE, Corvallis, OR (US)

(21) Appl. No.: 16/960,045

(22) PCT Filed: Mar. 23, 2018
(86) PCT No.: PCT/US2018/024047

§ 371 Date: Jul. 3, 2020

(51) Int. Cl. C12Q 1/686; G01N 21/64

(52) U.S. Cl. CPC C12Q 1/686; G01N 2021/6439; G01N 21/6428; G01N 21/6408

(57) ABSTRACT

An example method includes introducing a nucleic acid sample, a fluorescent indicator, and an internal positive control into a reaction volume of a microfluidic device. The example method further includes conducting a nucleic acid amplification reaction in the reaction volume, and optically monitoring a fluorescent signal from the reaction volume over a duration. A concentration of the internal positive control is selected to exhibit an initial increase in the fluorescent signal within the duration. The initial increase is to occur prior to a subsequent increase to be exhibited by an amplification product of a target when the target is present in the nucleic acid sample.

### BACKGROUND

Nucleic acid testing is often used to detect and identify microorganisms, such as pathogens that may cause disease in humans and animals. Nucleic acid testing is also used for forensic purposes, clinical studies, medical tests, and to conduct research.

Nucleic acid testing techniques may use nucleic acid amplification reactions, such as a polymerase chain reaction (PCR), real-time or quantitative polymerase chain reaction (qPCR), reverse transcription polymerase chain reaction (RT-PCR), loop mediated isothermal amplification (LAMP), and similar. In a typical amplification reaction, a template sequence of deoxyribonucleic acid (DNA) or ribonucleic acid (RNA) is copied multiple times, often by orders of magnitude in number, such that it is readily detectable.

### BRIEF DESCRIPTION OF THE DRAWINGS

FIG. 1 is a schematic diagram of an example device to perform a time multiplexed test on a nucleic acid sample using fluorescent signals.

FIG. 2 is a graph of example fluorescence over time in an example time multiplexed test of a nucleic acid sample using a fluorescent signal.

FIG. 3 is a flowchart of an example method to obtain information from a nucleic acid sample using time multiplexing with fluorescent signals.

FIG. 4 is a flowchart of an example method to perform a time multiplexed test on a nucleic acid sample with fluorescent signals.

FIG. 5 is a schematic diagram of an example system to perform a time multiplexed test on a nucleic acid sample using fluorescent signals.

FIG. 6 is a schematic diagram of an example device to perform temporal and spatial multiplexing using fluorescent signals.

FIG. 7 is a graph of example fluorescence over time for combined temporal and probe-based multiplexing using fluorescent signals.

FIG. 8 is a graph of example fluorescence over time in an example time multiplexed test of a nucleic acid sample using a fluorescent signal when a sequence of interest is not detected.

### DETAILED DESCRIPTION

Temporal multiplexing may be used to perform tests on samples containing nucleic acids. Temporal multiplexing may include sensing a plurality of fluorescent signals from a reaction volume at different times during the same test. Temporal multiplexing may reduce or eliminate the need to use spatial multiplexing, which often requires multiple reaction volumes and associated microfluidic structures and which may also need multiple optical sensing devices for the multiple reaction volumes. Temporal multiplexing may reduce or eliminate a need for custom reagents, such as fluorescent probes, when attempting to perform a test with different nucleic acid sequences in the same reaction volume. Hence, the quantity and complexity of microfluidic structures, sensors, and/or reagents may be reduced.

In a given test, target nucleic acid sequence may or may not be present in a sample. A fluorescent indicator may be provided to the sample to detect products of amplification during a DNA/RNA amplification process conducted within a reaction volume. Fluorescent signals exhibited by the fluorescent indicator may be optically detected at an optical sensing device. An internal positive control may also be provided to the sample. The internal positive control may have a concentration that is sufficiently high, so as to cause the fluorescent indicator to fluoresce before fluorescence that may occur as a result of amplification of the target nucleic acid sequence. Hence, an initial increase in a fluorescent signal is expected and detection of the initial increase may validate the test. Any subsequent increase in fluorescence may indicate the presence of the target DNA/RNA sequence in the sample. As such, sensing multiple fluorescent signals from the same reaction volume at different times during a test, i.e., temporal multiplexing, may be achieved.

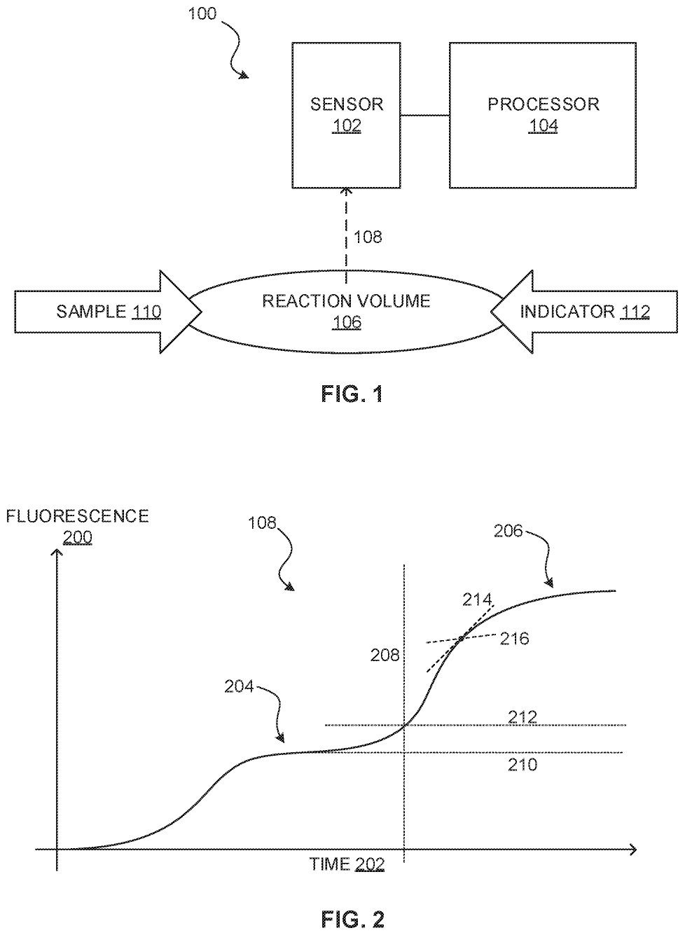

FIG. 1 shows an example device 100. The device 100 includes a sensor 102 and a processor 104. The device 100 is to perform a time multiplexed test on a nucleic acid sample, in that the test may indicate the presence of one or more DNA/RNA sequences in the sample over a duration of time.

The sensor 102 is positioned with respect to a reaction volume 106 to detect a fluorescent signal 108 emitted from within the reaction volume 106. The sensor 102 may include an optical sensor such as a photodiode, photodetector, or similar. The sensor 102 may be provided in a fluorometer.

The processor 104 may include a central processing unit (CPU), a microcontroller, a microprocessor, a processing core, a field-programmable gate array (FPGA), a graphics processing unit (GPU), or similar device capable of executing instructions. The processor 104 may cooperate with memory to execute instructions. Memory may include a non-transitory machine-readable storage medium that may be an electronic, magnetic, optical, or other physical storage device that stores executable instructions. The machine-readable storage medium may include, for example, random access memory (RAM), read-only memory (ROM), electrically-erasable programmable read-only memory (EEPROM), flash memory, a storage drive, an optical disc, and the like. The machine-readable storage medium may be encoded with executable instructions.

The reaction volume 106 may be provided in a microfluidic device. Such a microfluidic device may include the sensor 102, the processor 104, or both.

The reaction volume 106 may be the only reaction volume provided, as both a target sequence of interest and an internal positive control may be provided to the same reaction volume 106.

A nucleic acid sample 110 and a fluorescent indicator 112 may be provided to the reaction volume 106, so that a nucleic acid amplification reaction may be performed within the reaction volume 106. An amplification product of the nucleic acid amplification reaction may generate the fluorescent signal 108.

Examples of nucleic acid amplification reactions include PCR, qPCR, RT-PCR, LAMP, and similar. The nucleic acid amplification reaction may require temperature cycling, may be isothermal, or may have other conditions.

The device 100 may include an electromagnetic radiation source to excite fluorescence within the sample 110. The electromagnetic radiation source may be a light source or similar element provided in a fluorometer that contains the sensor 102.

A plurality of different nucleic acid sequences may be present in the nucleic acid sample 110 to act as templates in the amplification reaction.

A target nucleic acid sequence may or may not be present in the nucleic acid sample 110. That is, the device 100 may be used to test a sample 110 to determine whether or not a target nucleic acid sequence is present in the sample 110. In an illustrative example, the device 100 is used in food safety testing and the target nucleic acid sequence is a pathogen nucleic acid sequence, such as a Salmonella DNA sequence.

Another example nucleic acid sequence that may be present in the nucleic acid sample 110 is an internal positive control. An internal positive control may be provided to the nucleic acid sample 110 to validate that the nucleic acid amplification reaction occurs as expected. An internal positive control may be a nucleic acid sequence that is known to be expressed by the nucleic acid amplification reaction for the type of sample 110. For example, the internal positive control may be a synthetic DNA sequence that was created in a lab and that has been highly characterized.

A concentration of the internal positive control may be selected to be greater than a concentration of a target nucleic acid sequence that is anticipated to be present in the nucleic acid sample 110. The degree by which the concentration of the internal positive control is provided to exceed the anticipated concentration of the target nucleic acid sequence may be selected to sufficiently separate indications of the control and the target in the fluorescent signal 108, so that fluorescence from the control and any present target may be distinguished. For example, the concentration of the internal positive control may be at least five times greater than an anticipated concentration of the target nucleic acid sequence.

The fluorescent indicator 112 may be an intercalating dye, a generic fluorophore, a fluorescent probe, or similar. An intercalating dye produces a fluorescent signal 108 by insertion into the bases of a DNA/RNA sequence during amplification. Prior to insertion, little to no fluorescent signal 108 is generated by the dye. After insertion, which may be termed intercalation, the dye fluoresces and the fluorescent signal 108 produced may be proportional to the amount of nucleic acid product that was amplified. Intercalating dyes are not sequence-dependent, in that they do not discriminate between different sequences of DNA/RNA. Examples of fluorescent indicators 112 include ethidium bromide, propidium iodide, EvaGreen™, SYBR Green™, and similar.

In addition to the nucleic acid sample 110 and the fluorescent indicator 112, other components/reagents may be provided to the reaction volume, as required by the amplification reaction used. In the case of PCR, these additional components may include a buffer solution, DNA/RNA polymerase, primers, deoxyribonucleotide triphosphate (dNTP), a source of cations (magnesium, potassium, etc.), and similar.

The processor 104 is to monitor the fluorescent signal 108 via the sensor 102 during the nucleic acid amplification reaction that takes place within the reaction volume 106. The processor 104 is to indicate a plurality of increases in the fluorescent signal 108 with respect to time. A plurality or series of increases may be associated with a plurality of nucleic acid sequences present in a nucleic acid sample at different concentrations. Increases within a series of increases may be individually detectable. Increases may be indicated at an output device, such as a display, an illumination device (e.g., a light-emitting diode or LED), or similar. Increases in the fluorescent signal 108 or a representation of the signal 108 as a whole may be indicated graphically, numerically, textually, in a binary manner (e.g., by illumination of an LED), or in other ways.

FIG. 2 shows an example fluorescent signal 108. The fluorescent signal 108 may be representative of a fluorescence 200 over time 202 as measured by a sensor 102. Time 202 may be a representative cycle count, such as in the case of PCR and other thermal cycling amplification techniques.

An initial increase 204 in the fluorescent signal 108 may represent the detection of an internal positive control in the nucleic acid sample 110 by the sensor 102 and processor 104. The processor 104 may indicate the initial increase at an output device, such as by graphically representing the fluorescent signal 108, as shown in FIG. 2.

A subsequent increase 206 in the fluorescent signal 108 may be the detection of a target DNA/RNA sequence of interest in the nucleic acid sample by the sensor 102 and processor 104. Such a target nucleic acid sequence is expected to occur after the initial increase 204 when a concentration of the target nucleic acid sequence is less than a concentration of the internal positive control.

In PCR, a breakthrough or threshold cycle (Ct), as indicated at 208, for the target of interest may be determined with reference to a baseline fluorescence 210 determined from the initial increase 204. That is, a baseline fluorescence 210 may be computed based on a determined fluorescence of an initial increase 204 and a threshold fluorescence 212 may be added to the baseline fluorescence 210 to detect the threshold cycle 208. The threshold cycle 208 may be approximately the same, i.e., occur at about the same time after commencement of the reaction, as that of a singleplex reaction or a reaction with only the target of interest. The threshold cycle 208 may be used to determine a concentration of the target originally present in the sample.

The degree by which the concentration of the internal positive control exceeds the concentration of the target nucleic acid sequence is exhibited in the fluorescent signal 108 as separation between the plateaus of the initial increase 204, and the subsequent increase 206. That is, a concentration for the internal positive control that is too low may cause the initial increase 204 to appear indistinguishable from an initial rise of a subsequent increase 206. A concentration for the internal positive control that is too high may cause the rise of the initial increase 204 to mask a lagging rise of a subsequent increase 206, and the increases 204, 206 may appear as one large plateau.

Detection of an increase 204, 206 may be performed by a slope computation, in which a slope 214 of a fluorescent signal 108 is computed and compared to a threshold slope 216. If the slope 214 of a fluorescent signal 108 is less than the threshold slope 216, then it may be determined that an increase 204, 206 has occurred and that any subsequent increase has not yet occurred. Such a computation may be implemented at the processor 104. An increase 204, 206 may be referred to as a saddle point, plateau, or peak.

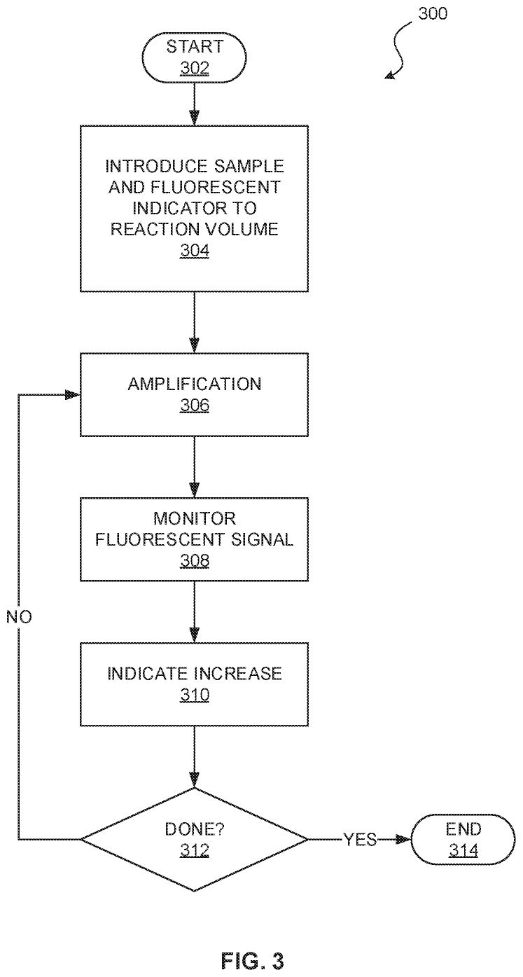

FIG. 3 shows an example method 300 to obtain information from a nucleic acid sample using time multiplexing. The method 300 may be performed by any of the devices and systems described herein. The method 300 starts at block 302.

At block 304, a nucleic acid sample and a fluorescent indicator are introduced to a reaction volume, such as a reaction volume in a microfluidic device. The nucleic acid sample may contain one or more different nucleic acid sequences, such as a target DNA/RNA sequence, an internal positive control, and the like.

At block 306, a nucleic acid amplification reaction is conducted in the reaction volume. For example, a PCR may be conducted to amplify the target sequence and the internal positive control.

At block 308, a fluorescent signal from the reaction volume is optically monitored over a duration of the amplification reaction. This may include using a sensor, such as that of a fluorometer, to measure fluorescence of the reaction volume.

At block 310, detected increases in the fluorescent signal may be indicated, for example, graphically, numerically, textually, in a binary manner (e.g., by illumination of an LED), or similarly. When the concentration of the internal positive control was selected to sufficiently exceed the concentration of the target nucleic acid sequence thought to be present in the sample, a first increase detected at block 310 validates the amplification reaction. Any subsequent increases may indicate the presence of the target nucleic acid sequence in the sample.

The monitoring and indicating, at blocks 308 and 310, may be performed continually as the amplification reaction occurs, as indicated by block 312. The method 300 may end at block 314 after, for example, a set time or set number of cycles.

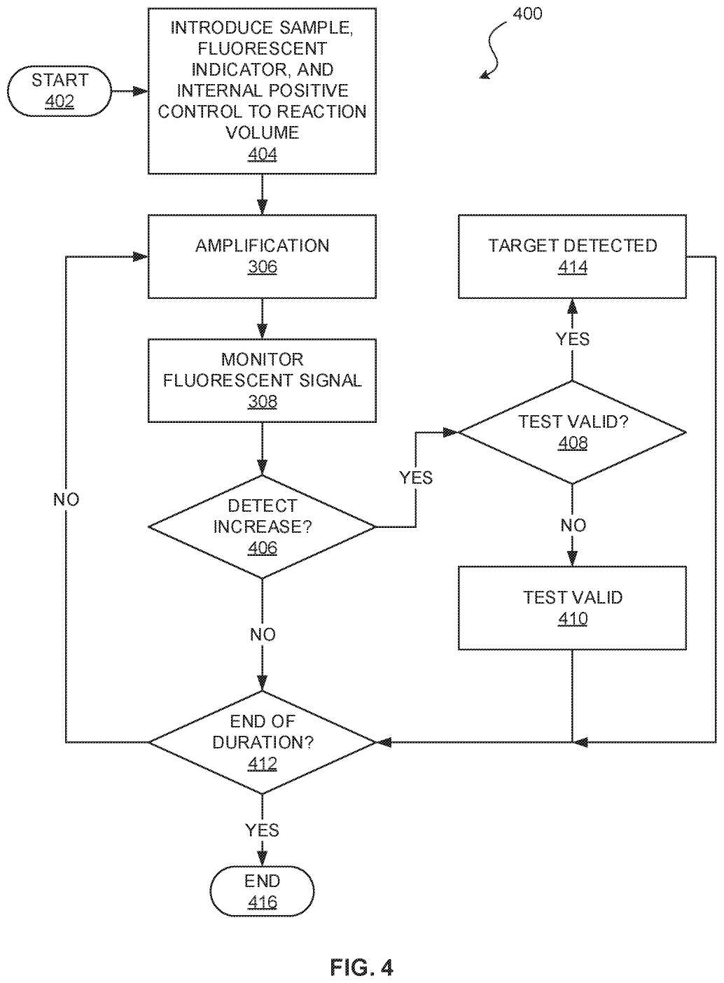

FIG. 4 shows an example method 400 to perform a time multiplexed test on a nucleic acid sample. The method 400 may be performed by any of the devices and systems described herein. The other methods discussed herein may be referenced for features and aspects not repeated here, with like reference numerals denoting like components. The method 400 starts at block 402.

At block 404, a nucleic acid sample, an internal positive control, and a fluorescent indicator are introduced to a reaction volume, such as a reaction volume in a microfluidic device. The method 400 may be to determine whether or not the nucleic acid sample contains a target DNA/RNA sequence. A concentration of the internal positive control may be selected to exhibit an initial increase in the fluorescent signal, so as to validate the amplification reaction, prior to any expected increase in the fluorescent signal due to the presence of the target DNA/RNA sequence.

The internal positive control may be mixed with the sample prior to amplification, may be provided ahead of time as stored in the reaction volume, or may be provided to the reaction volume separate from the sample, for example, through a separate microfluidic channel.

At block 306, a nucleic acid amplification reaction is conducted in the reaction volume, and at block 308, a fluorescent signal from the reaction volume is optically monitored.

At block 406, when an initial increase in the fluorescent signal is detected and the test has not yet been established to be valid, at block 408, then the test may be considered valid, at block 410.

The amplification, monitoring, and increase detection of blocks 306, 308, 406 may continue until a set duration, such as a set number of PCR cycles, is reached, as determined by block 412.

At block 406, if an increase is detected and the test has been established as valid due to prior detection of an increase in the fluorescent signal, at block 408, then the detected increase may be considered as the detection of the target sequence, at block 414.

The method 400 may end, at block 416, when the set duration has elapsed. If blocks 410 and 414 were reached, the test may be considered valid and the target sequence may be considered detected. If block 410 was reached and block 414 was not reached, then the test may be considered valid and the target sequence may be considered not detected. If block 410 was not reached, then the test may be considered invalid.

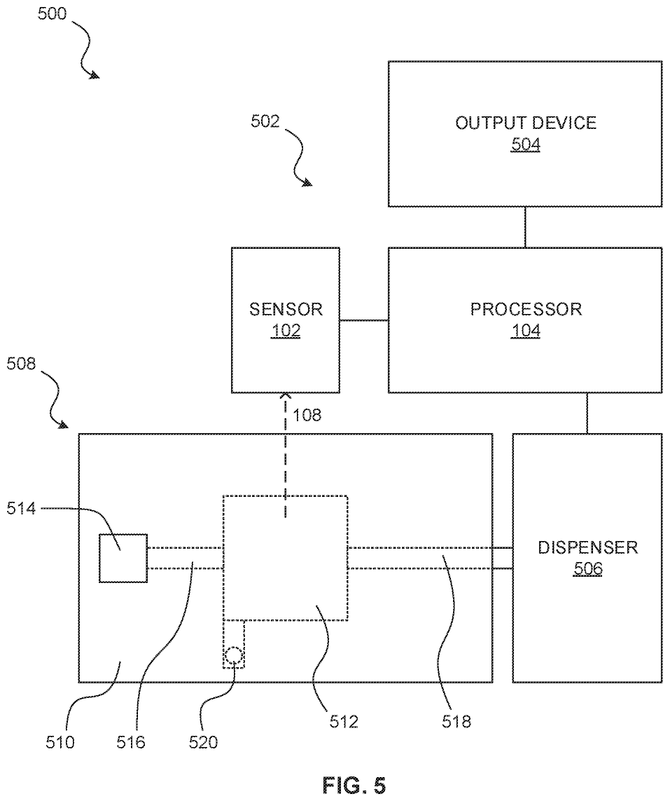

FIG. 5 shows an example system 500. The system 500 is to perform a time multiplexed test on a nucleic acid sample, in that the test may indicate the presence of one or more DNA/RNA sequences in the sample over a duration of time. The system 500 may include features and aspects of other devices and systems discussed herein, and the related description may be referenced, with like reference numerals denoting like components.

The system 500 includes a device 502 that includes a sensor 102 and a processor 104.

The device 502 may further include an output device 504 and a dispenser 506 connected to the processor 104.

The output device 504 may include a display, an illumination device (e.g., a light-emitting diode or LED), or similar. The output device 504 may output results of a test performed by the system 500, such as an indication that the test is valid and an indication of detection of a target sequence. In one example, the output device 504 includes an LED to indicate test validity and another LED to indicate target detection. In another example, the output device 504 includes a display to depict a graph of a fluorescent signal 108, such as shown in FIG. 2.

The dispenser 506 may include any number of reservoirs and channels to provide any necessary components/reagents required by the amplification reaction used. The dispenser 506 may be controlled by the processor 104 to dispense components/reagents.

The system 500 further includes a microfluidic device 508 at which the amplification reaction takes place. The microfluidic device 508 may include a substrate 510, such as silicon, glass, or similar. The substrate 510 may be referred to as a microfluidic chip. The microfluidic device 508 may further include a housing or frame to allow handling of the substrate 510. The housing or frame may further allow physical connection of the microfluidic device 508 to the device 502. The microfluidic device 508 may be a removable cassette, which may be one-time use.

A reaction volume 512 may be etched, molded, or otherwise provided to the substrate 510. The reaction volume 512 may include a window through which the sensor 102 may detect the fluorescent signal 108. The reaction volume 512 may be the only reaction volume provided, as both a target sequence of interest and an internal positive control may be provided to the same reaction volume 512.

A sample port 514 may be provided to the substrate 510 to receive a sample of material that may contain a target nucleic acid sequence. A sample reservoir may be provided in fluid communication with the sample port 514 to hold a sample received through the sample port 514. The sample port 514 may be an entranceway to the sample reservoir. An external sample cup may be provided in fluid communication with the sample port 514 to receive the sample from an external device. A microfluidic sample channel 516 may convey the sample to the reaction volume 512.

Any number of microfluidic component/reagent channels 518 may be provided to the substrate 510 to convey components/reagents from the dispenser 506 to the reaction volume 512.

An internal positive control may be provided to the reaction volume 512 through a microfluidic sample channel 516, a microfluidic component/reagent channel 518 connected to the dispenser 506, or another microfluidic channel provided to the substrate 510.

The microfluidic device 508 may further include a microfluidic flow device 520 to urge fluid into the reaction volume 512. For example, the microfluidic flow device 520 may include a thermal inject (TIJ) nozzle, a piezoelectric nozzle, or similar device that ejects fluid to create low pressure to draw fluid from the sample channel 516 and/or the component/reagent channel 518 into the reaction volume 512. The microfluidic flow device 520 may be driven by a signal from the processor 104.

The microfluidic device 508 may include micropumps, valves, mixers, and similar microfluidic elements and structures.

In other examples, the sensor 102, processor 104, and/or output device 504 may be provided as part of the microfluidic device 508. For example, the sensor 102, processor 104, and/or output device 504 may be mounted to the substrate 510.

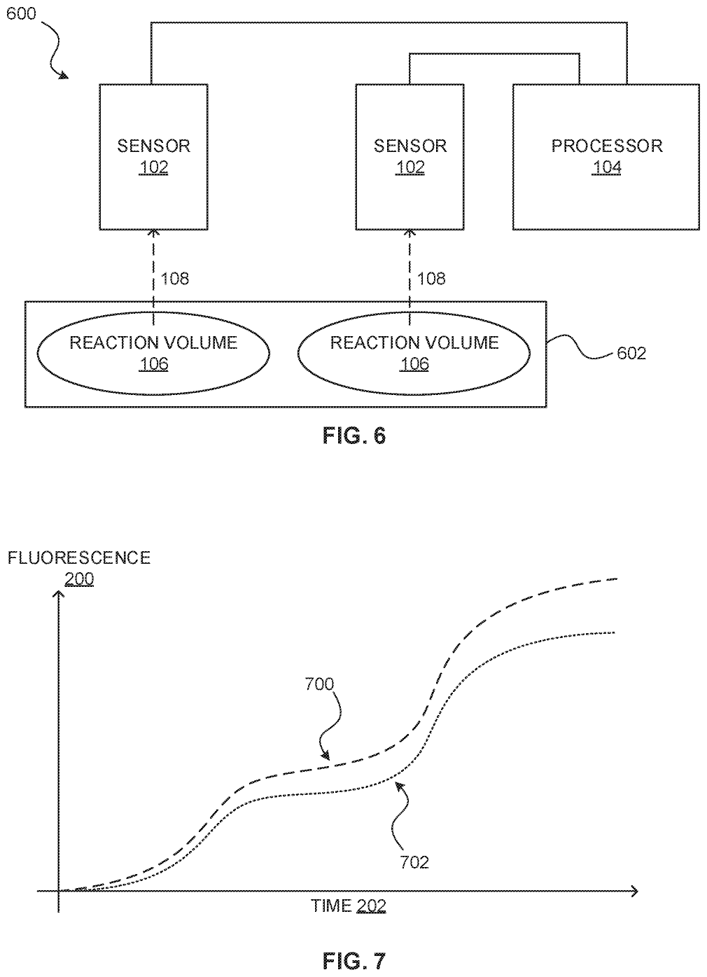

FIG. 6 shows an example device 600 to perform temporally and spatially multiplexed testing on nucleic acid samples. The device 600 may include features and aspects of other devices and systems discussed herein, and the related description may be referenced, with like reference numerals denoting like components.

The device 600 may include a plurality of sensors 102 to detect fluorescent signals 108 from a plurality of reaction volumes 106, which may be provided to a common substrate 602.

Spatial multiplexing using a plurality of reaction volumes 106 may be compatible with temporal multiplexing as described herein. Different nucleic acid tests or different components of the same test may be performed in different reaction volumes 106 at the same time that temporal multiplexing, as described herein, is performed in a reaction volume 106.

Temporal multiplexing may be realized with the inclusion of a plurality of target nucleic acid sequences within a reaction volume 106, such as a sequence being tested and an internal positive control at a concentration that significantly exceeds an expected concentration of the sequence being tested. The processor 104 may monitor the fluorescent signal 108 with respect to time. An initial increase in the fluorescent signal 108 may be due to the internal positive control and may indicate that a test is valid. Any subsequent increase in the fluorescent signal 108 may be due to the detection of a nucleic acid sequence of interest.

As shown in FIG. 7, temporal multiplexing as described herein may be used with probe-based multiplexing, when suitable probes are selected. That is, the same reaction volume may be used to test for a plurality of target sequences and associated controls. For example, a first target sequence and its positive control may be independently detectable from a second target sequence and its positive control by selecting suitable probes. A first probe may be selected to indicate the first positive control and the first target, and a second probe may be selected to indicate the second positive control and the second target. Any of the devices and systems described herein may be used with probe-based multiplexing.

Sequence-dependent oligonucleotides may be used to detect multiple target sequences, including respective positive controls, in the same reaction volume. Fluorescent signals 700, 702 of different wavelengths may be generated. These signals 700, 702 may be discriminated by the appropriate sensors. A first increase in a given signal 700, 702 represents proper amplification with any subsequent increase representing detection of a respective target of interest.

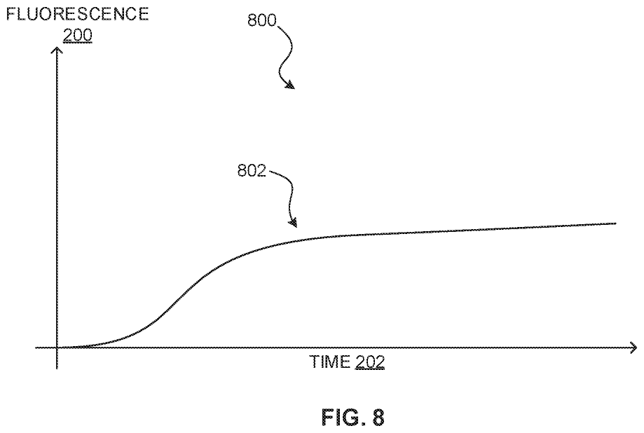

FIG. 8 shows an example fluorescent signal 800 when an initial increase 802 is detected but a subsequent increase is not detected. This may indicate that the amplification reaction proceeded as expected, with for example the initial increase 802 being due to the internal positive control, and that a target nucleic acid sequence of interest was not present in the sample. A signal of generally similar appearance may result if the concentration of the internal positive control is too high or too low. If too high, any increase in the signal due to presence of the target may be masked or not readily discernable. If too low, the increase 802 may actually be due to the presence of the target, with the signal from the positive control being masked or not readily discernable. Separation of increases in the signal due to the internal positive control and the target may be achieved with suitable selection of concentration of the internal positive control. A time component of the signal may be referenced to separate increases in the signal, as a threshold cycle or time for one or both of the internal positive control and the target may be predicable. That is, the time at which an initial increase occurs may be used to confirm that the increase is due to the internal positive control.

In view of the above, it should be apparent that the temporal multiplexing techniques described herein may allow for reduced complexity in microfluidic systems. Fewer reaction volumes and supporting microfluidic elements and structures, such as sample portioning microfluidics, may be required. In addition, reliance on custom reagents, such as fluorescent probes, may be reduced or eliminated.

It should be recognized that features and aspects of the various examples provided above can be combined into further examples that also fall within the scope of the present disclosure. In addition, the figures are not to scale and may have size and shape exaggerated for illustrative purposes.

1. A method comprising:
introducing a nucleic acid sample, a fluorescent indicator, and an internal positive control into a reaction volume of a microfluidic device;
conducting a nucleic acid amplification reaction in the reaction volume; and
optically monitoring a fluorescent signal from the reaction volume over a duration;
wherein a concentration of the internal positive control is selected to exhibit an initial increase in the fluorescent signal within the duration, the initial increase to occur prior to a subsequent increase to be exhibited by an amplification product of a target when the target is present in the nucleic acid sample.

2. The method of claim 1, wherein the concentration of the internal positive control is greater than a concentration of the target anticipated to be present in the nucleic acid sample.

3. The method of claim 2, wherein the concentration of the internal positive control is at least five times greater than a concentration of the target anticipated to be present in the nucleic acid sample.

4. The method of claim 1, further comprising detecting the initial increase in the fluorescent signal.

5. The method of claim 4, further comprising detecting the subsequent increase and indicating that the target is present in the nucleic acid sample.

6. The method of claim 4, wherein detecting the initial increase in the fluorescent signal comprises determining a slope of the fluorescent signal.

7. The method of claim 1, wherein the fluorescent indicator is an intercalating dye.

8. A device comprising:
a sensor to detect a fluorescent signal from a reaction volume; and
a processor to monitor the fluorescent signal during a nucleic acid amplification reaction in the reaction volume, the nucleic acid amplification reaction initiated with a nucleic acid sample and a fluorescent indicator to generate the fluorescent signal from an amplification product of the nucleic acid amplification reaction;
the processor to indicate a plurality of increases in the fluorescent signal with respect to time during the nucleic acid amplification reaction, the plurality of increases associated with a plurality of nucleic acid sequences present in the nucleic acid sample at different concentrations.

9. The device of claim 8, wherein the processor is to indicate an initial increase in the fluorescent signal as detection of an internal positive control.

10. The device of claim 9, wherein the processor is to indicate a subsequent increase in the fluorescent signal as detection of a target in the nucleic acid sample, the target having a concentration that is less than a concentration of the internal positive control.

11. The device of claim 8, wherein the processor is to determine increases in the fluorescent signal by comparing a slope of the fluorescent signal to a threshold slope.

12. The device of claim 8, wherein the processor is to determine a baseline fluorescence from an initial increase in the fluorescent signal to determine a subsequent increase in the fluorescent signal.

13. A system comprising:
a substrate defining a reaction volume;
a sensor to detect a fluorescent signal from the reaction volume; and
a processor to monitor the fluorescent signal during a nucleic acid amplification reaction in the reaction volume, the nucleic acid amplification reaction initiated with a nucleic acid sample and a fluorescent indicator to generate the fluorescent signal from an amplification product of the nucleic acid amplification reaction;
the processor to indicate a plurality of increases in the fluorescent signal with respect to time during the nucleic acid amplification reaction, the processor to indicate an increase of the plurality of increases as an internal positive control of the nucleic acid sample.

14. The system of claim 13, comprising a plurality of reaction volumes including the reaction volume and comprising a plurality of sensors including the sensor.

15. The system of claim 13, wherein the processor is to indicate an initial increase in the fluorescent signal as detection of the internal positive control, wherein the processor is to indicate a subsequent increase in the fluorescent signal as detection of a target in the nucleic acid sample, the internal positive control having a concentration that is greater than an anticipated concentration of the target.
